(19) United States
(12) Patent Application Publication
(10) Pub. No.: US 2014/0288617 A1
(43) Pub. Date: Sep. 25, 2014

(54) SYSTEMS AND METHODS FOR TREATING ATRIAL FIBRILLATION

(71) Applicant: Will Rosellini, Dallas, TX (US)

(72) Inventor: Will Rosellini, Dallas, TX (US)

(73) Assignee: REMOTE BIOMEDICAL LLC, Dallas, TX (US)

(21) Appl. No.: 14/210,555

(22) Filed: Mar. 14, 2014

Related U.S. Application Data
(60) Provisional application No. 61/884,727, filed on Sep. 30, 2013.
(60) Provisional application No. 61/784,913, filed on Mar. 14, 2013.

(51) Int. Cl. A61N 1/36; A61N 1/372

(52) U.S. Cl. CPC A61N 1/36139; A61N 1/37282; A61N 1/36053; A61N 1/36114
USPC 607/60; 607/62

(57) ABSTRACT

The present invention relates a method of treating heart failure in patients with coincident atrial fibrillation, the method comprising: screening of patients for selection of potential responders to neurostimulation based on heart rate variability; implanting a neurostimulator device around a vagus nerve in the selected patients followed by stimulating the vagus nerve at an electrical stimulus intensity below threshold for heart rate reduction; and remotely monitoring and controlling the neurostimulator based on cardiac health parameters of the patient subjected to vagal nerve stimulation.

### CROSS-REFERENCE TO RELATED APPLICATIONS

This application claims benefit of priority of U.S. Provisional Patent Application Ser. 61/884,727 filed on Sep. 30, 2013, titled “VAGUS NERVE STIMULATION FOR CONGESTIVE HEART FAILURE” which further claims the priority of No. 61/784,913, filed on Mar. 14, 2013, titled “METHODS AND SYSTEMS FOR SUPPRESSING ATRIAL FIBRILATION (AF)”, owned by the assignee of the present application and herein incorporated by reference in its entirety

### FIELD OF THE INVENTION

The present invention relates to systems and methods for treating cardiac dysfunction and, in particular, to an implantable neurostimulator for providing low level stimulus to a vagus nerve for the remote monitoring and treatment of heart failure in patients with coincident atrial fibrillation.

### BACKGROUND OF THE INVENTION

There are over 650,000 new cases of Congestive Heart Failure (CHF) each year in the United States. CHF is a serious, growing problem characterized by substantial morbidity and mortality. Vagus Nerve Stimulation (VNS) technology is known as an attractive potential solution for treating chronic heart failure (CHF). However, current therapies are limited in their ability to include patients with atrial fibrillation. In addition, current therapies do not integrate vital sign monitoring into a comprehensive remote monitoring and treatment paradigm. In response, we identified the areas needing growth and improvement and have built a plan to capitalize on using VNS technology for CHF treatment.

Atrial fibrillation (AF) is the most common cardiac arrhythmia, and is associated with substantial risk of morbidity and mortality. Atrial fibrillation occurs when the heart's electrical signals begin in another part of the atria or the pulmonary veins, rather than in the sino-atrial (SA) node. This leads to uncoordinated beating and incomplete emptying of the atria stemming from rapid and irregular atrial contractions. The ventricles also contract much faster than normal and do not efficiently pump blood through the circulatory system to the body.

Currently atrial fibrillation is estimated to affect more than 2.5 million Americans and may increase to as many as 12 million by 2050. It is a disease of aging, affecting 1 in 4 Americans over age 40, with further elevated risk for men and women above age 65. AF independently increases the risk of stroke by 4- to 5-fold, is an independent risk factor for stroke recurrence, and is responsible for at least 15-20% of all ischemic strokes. AF is associated with 50-90% increase in the risk of death. In addition to its associated health risks and diminished quality of life, AF is also a financial burden on the US Healthcare system, with annual Medicare costs for currently estimated at $16 billion.

Atrial fibrillation is a common heart condition in which the upper chambers, the atria, of the heart quiver instead of beating effectively. Rapid atrial beating produces a corresponding rapid beating of the ventricles. Electrical cardioversion and drugs have been used to restore the heart's normal rhythm. Chronic atrial fibrillation, in which a normal rhythm could not be restored, is commonly treated with medication, such as beta blockers, to slow the rapid heart rate.

The greatest danger to the atrial fibrillation (AF) patient is the significantly increased likelihood of a stroke, due to the tendency of clots to form in their poorly contracting atria. It is estimated that 20-25% of all strokes are caused by AF, and they are more severe than those caused by other factors. Other than strokes, the greatest risk to the AF patient is that rapid heart rate caused by AF can lead to cardiomyopathy and left ventricular dysfunction, which in turn can promote AF in a vicious cycle. Thus, more than 40% of individuals who experience AF will also experience congestive heart failure sometime in their lives. Even after accounting for such coexisting cardiovascular conditions, an individual with AF has an increased likelihood of premature death.

Atrial fibrillation can be treated by “rate” control (to reduce tachycardia) or “rhythm” control (to return patient's to a normal sinus rhythm) strategies. The optimal treatment strategy is often dependent on whether the patient's AF is classified as permanent, paroxysmal, or persistent. The use of antiarrhythmic drugs produces modest long-term reduction in AF recurrence, but is commonly associated with withdrawals due to adverse events, incidence of serious adverse events, and treatment discontinuation. Radiofrequency (RF) ablation has emerged as an alternative to antiarrhythmic medications for maintaining sinus rhythm. A systematic review of randomized trials indicated that select patients treated with RF ablation reduce their risk of AF recurrence at 1 year by 65% compared to patients treated with antiarrhythmic medications. However, in other studies, post-ablation follow-up at 5 or 6 years revealed success rates of only 29-55%. Success rates are also more favorable for patients with paroxysmal as opposed to either permanent or persistent AF. The lack of demonstrated long-term success, relative complexity, amount of myocardium destroyed, and overall complication rate associated with ablative techniques suggest the need for improvements to this approach.

Over the past decade, neuromodulation emerged as an alternative to antiarrhythmic medications and ablative procedures for the treatment of some forms of atrial fibrillation. The autonomic nervous system has substantial control over cardiac function, including the ability to modulate heart rate (chronotropy) conduction velocity (dromotropy), contraction (inotropy), and relaxation (lusitropy). Neural control of the heart is mediated by the cardiac autonomic nervous system (CANS), which is composed of the intrinsic-CANS (ICANS) and extrinsic-CANS (ECANS). The ICANS comprises axons and autonomic ganglia concentrated at the ganglionated plexi (GP) embedded within epicardial fat pads, whereas the ECANS consists of the soma in brain nuclei, vagosympathetic trunks, chains of ganglia along the spinal cord and the postganglionic axons that course en route to the heart. The ICANS is implicated in triggering focal AF arising from the pulmonary veins, but is under control of the ECANS.

Cervical vagal nerve stimulation started to be used clinically for treating drug-resistant epilepsy and potential usage is under investigation for treating depression and CHF among others. More than 10 years of experience in implanting cervical stimulating electrodes resulted in standardized surgical procedures. Common used electrodes have also been demonstrated sustainable from a neural tissue damage point of view. However, what has not reached a stable and optimal level yet is selectivity. In patients with coexisting cardiovascular conditions such as heart failure and atrial fibrillation, vagal nerve stimulation methods are not commonly employed as high levels of stimulation may exacerbate atrial fibrillation symptoms and leads to arrhythmia.

Low-level electrical vagosympathetic stimulation (LL-VNS), delivered during the atrial refractory period at intensities 10-50% below that which slows the sinus rate, inhibits AF inducibility, likely by suppressing activity of the ICANS and stellate ganglion.

However, to date the majority of studies investigating LL-VNS as a treatment for AF were conducted in open procedures in anesthetized dogs. Recently, Shen et al., showed LL-VNS suppresses stellate ganglion activity and reduces atrial tachyarrhythmias in ambulatory dogs. In this study, a bipolar pacing lead was sutured around the left vagus nerve and connected to a subcutaneous neurostimulator. A radiotransmiter was also implanted to record activity from the left stellate ganglion, left vagus nerve, and left gabglionated plexi. A microcontroller was recently added to this implantable system for interactive stimulation based on left stellate ganglion activity; however, results of the system's performance are not yet available.

U.S. Pat. No. 8,005,542 describes therapeutic maintenance of atrial fibrillation by electrical stimulation of vagus nerve. U.S. Pat. No. 8,386,056 relates to method of parasympathetic nerve stimulation for treating atrial arrhythmia and heart failure. US patent publication US20060095081 A1 describes method and apparatus for sensing cardiac activity via neurological stimulation therapy system. US patent publication US20130131746 shows non-invasive vagus nerve stimulation devices and methods to treat or avert atrial fibrillation.

Tosato et al., showed activation of laryngeal intrinsic muscles could be greatly diminished by anodal block: using a symmetrical tripolar cuff, quasitrapezoildal pulses were used to squelch the Aβ fiber contribution to the Vagal Compound Action Potential (VCAP) hence preventing RLN stimulation. Note that in order to compensate for anodal contacts differences hence ensuring minimal current leakage and prevent anodal break 2 independent stimulators were used.

Vuckovic et al., compared the dimensional/directional selectivity techniques achievable with a single generator in vitro. Using a tripolar cuff on vagus samples submerged in Krebs solution selective large myelinated fibers blockade was attempted using quasitrapezoidal pulses, subthreshold square and slowly rising prepulses. While all techniques showed fit for blocking, subthreshold square prepulses, originally proposed by Deurloo and Grill inject the least charge. At the same time they are also extremely sensitive to conditions and parameters, and have failed to exert selective stimulation in a comparable theoretical study.

Ordelman et al., used a multicontact cuff and demonstrated the possibility to exert maximal effects on different cardiovascular parameters depending on the site of stimulation. Bipolar stimulation was applied to 2 out of 4 possible 90° spaced contacts in the cuff, giving 4 possible configurations, further compared to standard tripolar ring cuff stimulation. Their result support the hypothesis that functionally different cardiac fibers are distributed among the fascicles, so that VNS will mostly influence heart rate or left ventricular pressure depending on where it is applied. No mention on effects on other end organs. Rozman et al published a short paper presenting a 99 multicontact cuff for VNS.

Anholt et al., demonstrated the selectivity achievable with the Biocontrol VNS paradigm in vitro: combining the particular contacts configuration of the CSL and quasitrapezoidal pulses the Aβ fiber contribution to VCAP was diminished. Presented results are similar to Tosato et al. but with lower pulse amplitude and duration, and using a single generator: conditions in vitro are usually different from those in vivo.

However, the systems for treating heart failure patients including atrial fibrillation patients using neurostimulation discussed in the prior art normally employs implantable neurostimulator device that includes a power source, pacing leads, implanted pulse generator and larger external circuitry thus resulting in increased size and complexity during implantation process. Further limitations of the present techniques of neurostimulation includes selective stimulation of nerve fibers, complicated side effects arising from stimulation of untargeted nerves, lack of control over restricted current flow and lack of greater physiological control.

Therefore, there still exists a need in the art for a non-pharmacological, non-ablative modality for the treatment of heart failure in patients with atrial fibrillation. In specific, treatment by vagal nerve stimulation with reduced side effects, ease of implantation and granting greater physiological control.

### SUMMARY OF THE INVENTION

The present invention discloses systems and methods for treating heart failure in patients with coincident atrial fibrillation by low level vagosympathetic nervous stimulation using an implantable medical device that can be monitored and controlled from a remote location by a healthcare provider. The implantable medical device comprises a neurostimulator implanted around a vagal nerve which provides electrical stimulus delivery at intensity below the threshold needed for heart rate reduction.

In one embodiment of the present invention, the system for treating heart failure in patients with atrial fibrillation comprises i) a screening module for pre-selection of patients based on cardiac health parameters; ii) an implantable medical device configured to deliver low level electrical pulses for stimulation of a parasympathetic nerve and iii) a monitoring module configured to monitor changes in cardiac health parameters of patients.

In another embodiment, the implantable medical device comprises a stimulation device integrated with an electrode in contact with the vagal nerve and is in bi-directional communication with a communication module located external to the patient's body. The implantable medical device further comprises a memory and a software component. The communication module comprises a remote computer system for updating and reprogramming of the software component in the implantable medical device.

A further embodiment of the present invention provides a method for treating cardiac dysfunction, comprising steps of i) screening of patients for neurostimulation based on cardiac parameters including heart rate variability; ii) implanting a neuro stimulator around a vagus nerve the patient; iii) stimulating the vagus nerve at an electrical stimulus intensity below threshold that is required for heart rate reduction; and iv) remotely monitoring and controlling the neurostimulator based on cardiac health parameters of the patient.

Still other embodiments of the present invention will become readily apparent to those skilled in the art from the following detailed description, wherein are described embodiments by way of illustrating the best mode contemplated for carrying out the invention. As will be realized, the invention is capable of other and different embodiments and its several details are capable of modifications in various obvious respects, all without departing from the spirit and the scope of the present invention. Accordingly, the drawings and detailed description are to be regarded as illustrative in nature and not as restrictive.

### BRIEF DESCRIPTION OF THE DRAWINGS

FIG. 1 illustrates a block diagram of a system for treating heart failure in patients with atrial fibrillation according to an embodiment of the present invention.

### DETAILED DESCRIPTION OF THE INVENTION

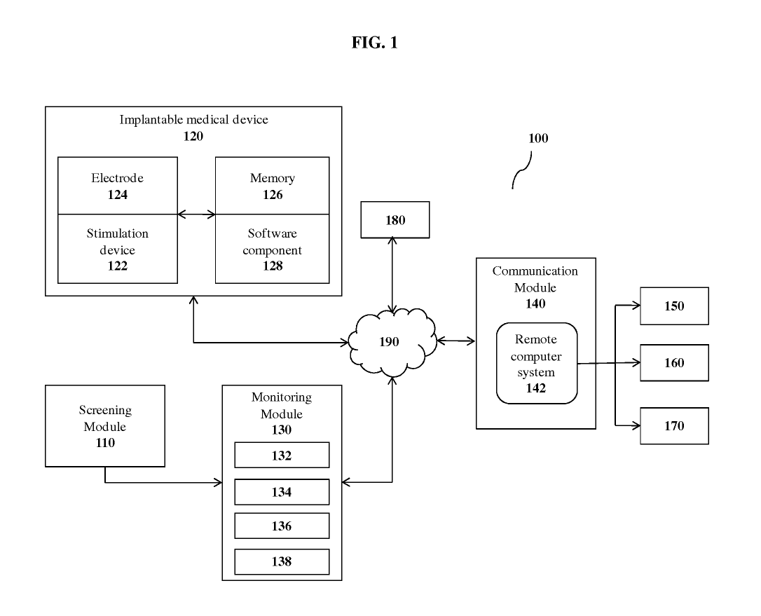

FIG. 1 is a block diagram that schematically illustrates a system 100 for treating heart failure patients with coincident atrial fibrillation in accordance with an embodiment of the present invention. The system comprises a screening module 110 for pre-selection of patients for neurostimulator implantation based on cardiac health parameters such as heart rate. The system further comprises an implantable medical device 120 such as a neurostimulator device implanted around a portion of a vagus nerve for delivering low level electrical pulses and a monitoring module 130 for monitoring cardiac health parameters including heart rate, pulse rate, blood oxygen saturation level and body weight of a patient subjected to implantation.

The implantable medical device 120 comprises a stimulation device 122 integrated with an electrode 124. The simulation device 122 generates electrical pulses that are transferred to the electrode 124 implanted around a portion of left vagus nerve. Electrical stimulus is selected at intensity below the threshold level that is required for slowing sinus rate or atrioventricular conduction or heart rate in general. The implantable medical device 120 further comprises a memory 126 for storing stimulation parameters as pulse width, amplitude, time interval, repetition rate and a software component 128, in bidirectional communication with a communication module 140 located external to patient's body or in a remote location.

The communication module 140 comprises a remote computer system 142, which in turn comprises a means for upgrading and reprogramming of the software component 128 in the implantable medical device 120. In addition, the remote computer system 142 is connected to a mobile network 180 through a communication network 190, wherein the mobile network 180 is capable of receiving information regarding the software component 128 of the implantable medical device 120 and relaying the information to the remote computer system 142. In an embodiment of the present invention, the remote computer system 142 is operatively connected to a healthcare provider 150 thereby allowing remote monitoring or diagnosis or treatment of implanted patients by the healthcare provider 150; an automatic expert system 160 suggesting appropriate remedial action based on cardiac health parameters of the patient; and an emergency control room 170 for requesting dispatch of emergency medical help.

In an embodiment, the monitoring module 130 comprises physiological sensors monitoring cardiac health parameters of implanted patients. The sensors comprises a heart rate sensor 132, a pulse rate sensor 134, a blood oxygen saturation level (SpO2) sensor 136 or a body weight sensor 138, all of which are configured to communicate with a remote computer system 142 through the communication network 190 such as internet network.

The monitoring module may comprise additional physiological sensors including, but are not limited to, pressure-volume (PV) loops, pressure-area (PA) loops, pressure-dimension (PD) loops, diastolic and systolic pressures, estimated pulmonary artery pressure, change in cardiac pulse pressure, pre-ejection timing intervals, heart rate measures (such as, rates, intervals, and the like), autonomic indicators (such as, heart rate variability, direct neural recordings, and the like), chemical sensors (such as, catecholamines, O2 (saturated venous and/or arterial), pH, CO2, and the like), or non-cardiac physiologic sensors (such as, activity, respiratory rate, time of day, posture, and the like).

In another embodiment, the stimulation device 122 comprises a wireless bipolar stimulation device that is configured to receive stimulus from an external pulse generator. In a further embodiment, the bipolar stimulation device generates electrical current that is conducted to the vagus nerve through a pacing lead insulated with silicone and trifurcated in order to provide bipolar or tripolar stimulation.

A further embodiment of the present invention provides a method for treating cardiac dysfunction, comprising steps of i) screening of patients for neurostimulation based on cardiac parameters including heart rate variability; ii) implanting a neuro stimulator around a vagus nerve in the patient; iii) stimulating the vagus nerve at an electrical stimulus intensity below threshold that is required for heart rate reduction; and iv) remotely monitoring and controlling the neurostimulator based on cardiac health parameters of the patient.

The neurostimulator 120 implanted in the patient establishes communication with a remote computer system 142 which is connected to a health care provider allows remote monitoring of the neuro stimulator 120 and providing treatment options by the healthcare provider. In addition to remote monitoring of neurostimulator 120, the remote computer system 142 comprises a means for generating invoice for the diagnosis or treatment provided to the patient by the healthcare provider.

Heart rate reduction threshold is identified as the electrical stimulus intensity required for reducing sinus rate or atrioventricular conduction. For vagal nerve stimulation, electrical stimulus that is 10-80% below the heart rate reduction threshold shows marked reduction in atrial fibrillation inducibility.

In an embodiment, the electrode 124 comprises a bipolar cuff electrode that creates a physical boundary to current leakage and this makes waveform related blocking techniques more effective. Cuffs electrodes with longer distance between cathode and anode widens the window of large fibers block and thin fibers excitation, while keeping current and pulse duration as low as possible. Cuffs made of biocompatible and softer material such as Teflon avoids mechanical damage to the neural tissue and providing a loose surgical mesh could also strengthen the structure.

In an embodiment, electrode 124 comprises two anode-cathode pairs(one for stimulating, one for blocking) connected to two independent pulse generator or same pulse generator resulting in stimulation by one pair of electrode (current pulses) and high frequency block with the other pair of electrode (sinusoidal). In another embodiment, a bipolar cuff electrode comprises a few pairs of point cathode with circular anode around the cathode positioned evenly around the cuff would minimize current leakage and allows precise selectivity of inner fascicles.

Vagal nerve stimulation elicits bi-directional activation of both afferent and efferent nerve fibers. The balance between achieving therapeutic benefits (afferent) and side-effects (efferent) is largely determined by the threshold differences between activation of the different vagus nerve fibers. Vagal nerve stimulation can be unilateral type involving either left vagal nerve or right vagal nerve and bilateral type stimulation involving stimulation of patient's vagus nerve by synchronously or asynchronously applying a stimulating electrical signal the right and left vagal nerves.

In a further embodiment, the neurostimulator 120 provides continuous alternating ON-OFF cycles of vagal stimulation that when applied to the vagus nerve through the bipolar cuff electrodes 124, produce action potentials in the underlying nerves that propagate bi-directionally; afferently propagating action potentials activate the medial medullary sites responsible for central reflex control and efferently propagating action potentials activate the heart's intrinsic nervous system. Cardiac motor neurons, when activated, influence heart rate, AV nodal conduction, and atrial and ventricular inotropy, thereby providing chronic cardiac dysfunction therapeutic effects. In addition, the alternating cycles can be tuned to activate phasic parasympathetic response in the vagus nerve being stimulated by bi-directionally modulating vagal tone.

In an embodiment, the remote computer system 142 provides automatic feedback to the implantable medical device or neurostimulator 120 in a closed loop control system based on the physiological data received from the monitoring module. Those skilled in the art will appreciate that any of a wide variety of measurable physiologic parameters may be monitored and used to implement the closed-loop control system described herein.

In an exemplary embodiment, the neurostimulator 120 implanted in a patient sends stimulation parameter related data to the remote computer system 142, wherein the remote computer system also receives cardiac health parameters related data from the physiological sensors 132, 134, 136, 138 and in response sends appropriate control signals to the neurostimulator 120 based on changes in cardiac health parameters. The remote computer system can provide access to a healthcare provider 150 for providing treatment from a remote location, or an emergency control room 160 for dispatching emergency medical care in case of critical conditions, or an automatic expert system 170 suggesting remedial action based on cardiac health parameters.

What is claimed is:

1. A system for treating heart failure in patients with coincident atrial fibrillation, comprising:
a screening module for selecting patients for implantation based on predetermined cardiac health parameters;
an implantable medical device configured to deliver low level electrical pulses to a parasympathetic nerve; and
a monitoring module configured to monitor changes in cardiac health parameters of the patient which enables remote treatment configuration of the device

2. The system of claim 1, wherein the implantable medical device comprises a stimulation device integrated with an electrode in contact with parasympathetic nerve.

3. The system of claim 1, wherein the implantable medical device is in bi-directional communication with a communication module located external to the patient's body.

4. The system of claim 2, wherein the implantable medical device further comprises a memory and a software component.

5. The system of claim 3, wherein the communication module comprises a remote computer system, wherein the remote computer system comprises a means for updating and reprogramming of the software component in the implantable medical device.

6. The system of claim 5 further comprises a communication network coupling the remote computer system to the communication module through a mobile network.

7. The system of claim 5, wherein the remote computer system is connected to an automatic expert system configured to render suggested course of therapeutic action.

8. The system of claim 5, wherein the remote computer system is connected to a healthcare provider allowing remote monitoring, diagnosis and treatment.

9. The system of claim 2, wherein the stimulation device is configured to receive a stimulus from an external pulse generator.

10. The system of claim 1, wherein the parasympathetic nerve comprises a vagus nerve.

11. The system of claim 1, wherein the electrical pulses are delivered at a rate below heart rate reduction threshold.

12. The system of claim 1, wherein the cardiac health parameters comprises of heart rate, blood pressure, blood oxygen saturation level, body weight or pulse rate.

13. The system of claim 1, wherein the screening module comprises a device configured to measure heart rate variability.

14. The system of claim 1, wherein the monitoring module is connected to the remote computer system through the communication network.

15. A method of treating heart failure in patients with atrial fibrillation, the method comprising:
screening of a patient for neurostimulation based on heart rate variability;
implanting a neurostimulator around a vagus nerve in the patient;
stimulating the vagus nerve at an electrical stimulus intensity below threshold for heart rate reduction;
monitoring cardiac health parameters of the patient; and
remotely controlling the neurostimulator based on changes in cardiac health parameters.

16. The method of claim 15, wherein the neurostimulator comprises a stimulation device integrated with an electrode.

17. The method of claim 15, wherein the cardiac health parameters comprises of heart rate, blood pressure, blood oxygen level, body weight or pulse rate.

18. The method of claim 15, wherein the electrical stimulus intensity is 10 to 80 percent below threshold for heart rate reduction.

19. The method of claim 15, wherein the neurostimulator is in bidirectional communication with a remote healthcare provider.

20. The method of claim 15, wherein the cardiac health parameters are remotely monitored by the remote healthcare provider.
